(19) United States
(12) Patent Application Publication
(10) Pub. No.: US 2020/0013495 A1
(43) Pub. Date: Jan. 9, 2020

(54) PORTABLE MEDICAL DEVICE AND CONTROL METHOD OF PORTABLE MEDICAL DEVICE

(71) Applicant: TERUMO KABUSHIKI KAISHA, Tokyo (JP)

(72) Inventor: Yu TORAI, Hadano-shi (JP)

(73) Assignee: TERUMO KABUSHIKI KAISHA, Tokyo (JP)

(21) Appl. No.: 16/578,068

(22) Filed: Sep. 20, 2019

Related U.S. Application Data
(63) Continuation of application No. PCT/JP2018/003217, filed on Jan. 31, 2018.

(30) Foreign Application Priority Data
Mar. 23, 2017 (JP) 2017-056942

(51) Int. Cl. G16H 20/17; H04W 76/19

(52) U.S. Cl. CPC G16H 20/17; H04W 76/19

(57) ABSTRACT

A portable medical device includes: a device body including: a power supply unit, a driving unit configured to be driven by electric power from the power supply unit, a main-body-side wireless communication unit configured to output identification information at a regular interval when the power supply unit is turned on, and a main-body-side input/output control unit configured to control driving of the driving unit and the output of the main-body-side wireless communication unit, in accordance with information received by the main-body-side wireless communication unit; and a remote controller configured to operate driving of the device body, which includes: an input unit configured to receive a stop request for a stop of the output from the main-body-side wireless communication unit, and at least one of a stop duration of the stop and/or a restart time at which the output is to be restarted, and a controller-side wireless communication unit.

### CROSS-REFERENCE TO RELATED APPLICATIONS

This is a bypass continuation of PCT Application No. PCT/JP2018/003217, filed on Jan. 31, 2018, which claims priority to Japanese Application No. 2017-056942, filed on Mar. 23, 2017. The contents of these applications are hereby incorporated by reference in their entireties.

### BACKGROUND

The present disclosure relates to a portable medical device and a control method of the portable medical device.

As a treatment for diabetic patients, a treatment of continuously infusing a small amount of insulin into the patient's body is performed. In this treatment, in order to continuously administer a drug solution (insulin) to a patient, there is used a portable drug solution administration device (so-called insulin pump) provided with a device body that is fixed to the patient's body or clothes to be used, and a remote controller to control the device body through wireless communication.

In order to reduce battery consumption on the device body side and reduce its required battery capacity, such a portable medical device using wireless communication has a configuration in which wireless communication is started, with a transmission signal from the device body as a trigger, by the remote controller receiving the transmission signal, at the time of wireless communication. Furthermore, the wireless communication between the device body and the remote controller can be interrupted by setting a communication setting between the device body and the remote controller to an in-flight mode when the user boards an airplane (see U.S. Pat. Pub. No. 2016/0,038,671 A).

### SUMMARY

However, in a case in which no user interface is provided on the device body, the portable medical device configured as described above cannot restart the wireless communication without an operation of the remote controller, after the wireless communication between the device body and the remote controller is once interrupted. Therefore, although drug solution administration by the device body is continued, it becomes impossible to perform control such as setting change of administration and additional drug solution administration.

Accordingly, an object of certain embodiments of the present disclosure is to provide a portable medical device and a control method of the portable medical device capable of restarting wireless communication even without an operation of a remote controller, even when wireless communication between a device body having no user interface and the remote controller to control the device body is stopped.

According to one embodiment, a portable medical device includes a device body and a remote controller that operates driving of the device body. The device body includes: a power supply unit; a driving unit driven by electric power from the power supply unit; a main-body-side wireless communication unit that outputs identification information at a regular interval when the power supply unit is turned on; and a main-body-side input/output control unit that controls driving of the driving unit and output of the main-body-side wireless communication unit, in accordance with information received by the main-body-side wireless communication unit. The remote controller includes: an input unit that is inputted with, together with a stop request of output for the main-body-side wireless communication unit, a stop duration of the output or a restart time of the output; and a controller-side wireless communication unit that establishes wireless connection with the main-body-side wireless communication unit that has outputted the identification information by receiving the identification information outputted from the main-body-side wireless communication unit, and outputs, to the main-body-side wireless communication unit, information that is related to stop of the output and inputted from the input unit.

According to certain embodiments of the present invention having the configuration above, it becomes possible to restart wireless communication without an operation of the remote controller, even when wireless communication between the device body having no user interface and the remote controller to control the device body is stopped.

### BRIEF DESCRIPTION OF THE DRAWINGS

FIG. 1 is a schematic configuration view of a portable medical device of a first embodiment.

FIG. 2 is a block diagram for explaining the portable medical device of the first embodiment.

FIG. 3 is a flowchart (part 1) for explaining a control method of the portable medical device of the first embodiment.

FIG. 4 is a flowchart (part 2) for explaining the control method of the portable medical device of the first embodiment.

FIG. 5 is a time chart for explaining a state of wireless communication of the portable medical device of the first embodiment.

FIG. 6 is a schematic configuration view of a portable medical device of a second embodiment.

FIG. 7 is a block diagram for explaining the portable medical device of the second embodiment.

FIG. 8 is a flowchart for explaining a control method of the portable medical device of the second embodiment.

### DETAILED DESCRIPTION

Hereinafter, embodiments of the present invention will be described in detail with reference to the drawings. Note that, in each embodiment, a description is given of a configuration in which the present invention is applied to a drug solution administration device, such as an insulin pump, as a portable medical device. However, the portable medical device of the present invention can be widely applied to portable medical devices that include a device body and a remote controller to operate or monitor this device body through wireless communication, for example, a pain relief device or a biological information measurement device. Further, without limiting to the device body having no user interface as in the embodiment, even a device body having a user interface can restart wireless communication without performing an input operation.

### First Embodiment

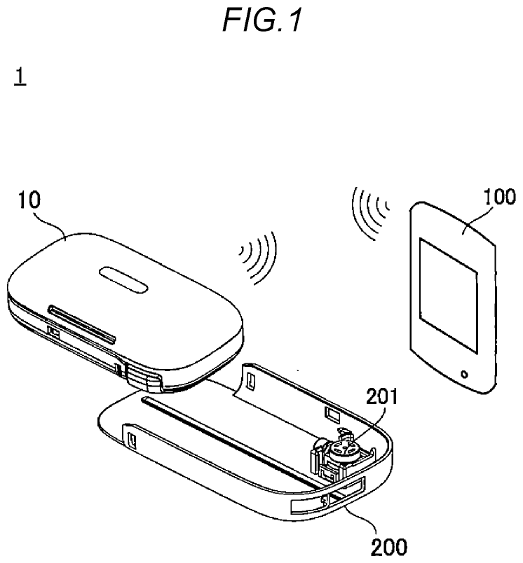

FIG. 1 is a schematic configuration view of a portable medical device of a first embodiment. A portable medical device 1 of the first embodiment shown in FIG. 1 includes a device body 10 and a remote controller 100 to operate the device body 10 through wireless communication.

Among them, the device body 10 is a pump unit to administrate a drug solution (insulin, as an example) to a patient for an extended time, and is used by being worn on the skin of the patient via a cradle 200 that is to be bonded to the skin. Note that the cradle 200 includes a catheter port 201, and is used by being bonded to the skin of the patient, whereas the remote controller 100 is carried and used without being worn on the skin of the patient. Such a remote controller 100 is configured as a central device (master device) for operation of the device body 10 through wireless communication, when the device body 10 is a peripheral device (slave device).

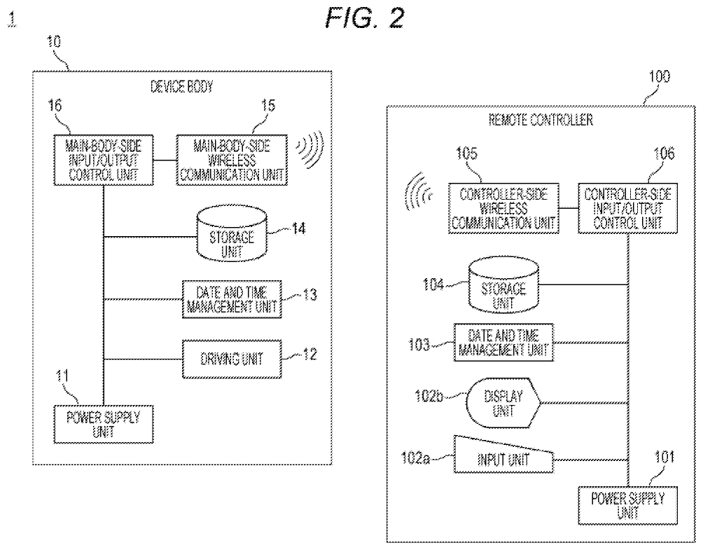

FIG. 2 is a block diagram for explaining the portable medical device of the first embodiment. Hereinafter, with reference to FIG. 2, details of a configuration of the portable medical device 1 will be described in the order of the device body 10 and the remote controller 100.

<Device Body 10>

In the device body 10, elements for performing administration of a drug solution (insulin as an example) are arranged. Such a device body 10 includes a power supply unit 11, a driving unit 12, a date and time management unit 13, a storage unit 14, a main-body-side wireless communication unit 15, and a main-body-side input/output control unit 16 that controls these. However, the device body 10 is configured to have no user interface for operation of the driving unit 12 or the main-body-side wireless communication unit 15. Hereinafter, the elements constituting the device body 10 are described.

[Power Supply Unit 11]

The power supply unit 11 is for supplying electric power to each component constituting the device body 10, and includes, for example, a battery and a battery box to accommodate the battery. The device body 10 is configured such that power is turned on by, for example, mounting the battery to the battery box.

[Driving Unit 12]

The driving unit 12 is for performing a drug solution administration to a patient wearing the device body 10, and includes a syringe to contain a drug solution to be administered, a pusher to be slid in the syringe, a motor and a gear mechanism for movement of the pusher, and the like. This driving unit 12 is configured to drive the motor by electric power from the power supply unit 11.

[Date and Time Management Unit 13]

The date and time management unit 13 is a program part to perform date and time management, which may be installed in a microcomputer, and outputs date and time information.

[Storage Unit 14]

The storage unit 14 is a part to store various data. Data stored in the storage unit 14 includes, for example, an administration condition of a drug solution transmitted from the remote controller 100 side, a wireless output stop setting, an administration history of a drug solution administration performed by the device body 10 and the like.

[Main-Body-Side Wireless Communication Unit 15]

The main-body-side wireless communication unit 15 is a part that performs wireless communication with the remote controller 100. This main-body-side wireless communication unit 15 is a part that has a transmitter and a receiver and performs wireless communication using radio waves of a predetermined wavelength band, and in particular, performs wireless communication using ultra high frequency waves or microwaves. In the main-body-side wireless communication unit 15, short-distance wireless communication with a communication distance of 100 m or less is to be performed. Specifically, a standardized wireless communication technology such as ZigBee® (IEEE 802.15.4: frequency 868 MHz band, 915 MHz band, 2.4 GHz band), Bluetooth® (IEEE 802.15.1: frequency 2.4 GHz band), or wireless LAN (IEEE 802.11a/b/g/n/ac: frequency 2.4 GHz band, 5 GHz band) is applied.

In particular, the main-body-side wireless communication unit 15 is configured to periodically transmit identification information for causing the remote controller 100 to recognize the device body 10. The device body 10 is configured to have no user interface for operation of the main-body-side wireless communication unit 15. Therefore, output of identification information by such a main-body-side wireless communication unit 15 is to be started with turning on of the power supply unit 11 as a trigger.

Further, such a main-body-side wireless communication unit 15 performs transmission and reception of signals with the controller-side wireless communication unit 105 of the remote controller 100 that has received the identification information, and the main-body-side wireless communication unit 15 establishes connection for wireless communication with the controller-side wireless communication unit 105.

Furthermore, in accordance with an instruction from the main-body-side input/output control unit 16, the main-body-side wireless communication unit 15 performs transmission and reception of an administration condition of a drug solution, a wireless output stop setting, an administration history of drug solution administration, and the like, with the remote controller 100 with which the wireless communication connection has been established.

Such a main-body-side wireless communication unit 15 may be any one that performs wireless communication connection in the above-described procedure. For example, wireless communication using Bluetooth Low Energy® may be performed.

Further, the device body 10 provided with such a main-body-side wireless communication unit 15 is configured as, with the remote controller 100 as a sensor device, a beacon device that transmits identification information of the device body 10 to the sensor device.

[Main-Body-Side Input/Output Control Unit 16]

The main-body-side input/output control unit 16 is connected to the power supply unit 11, the driving unit 12, the date and time management unit 13, the storage unit 14, and the main-body-side wireless communication unit 15. This main-body-side input/output control unit 16 controls input and output of signals between these units, and controls drug solution administration by driving of the driving unit 12 and wireless communication by the main-body-side wireless communication unit 15, in accordance with information received by the main-body-side wireless communication unit 15.

Such a main-body-side input/output control unit 16 includes, for example, a central processing unit (CPU), a random access memory (RAM), and a read only memory (ROM), which are not shown here. The read only memory (ROM) may be the storage unit 14. An algorithm executed by such a main-body-side input/output control unit 16 for the control of drug solution administration from the driving unit 12 and wireless communication by the main-body-side wireless communication unit 15 will be described in detail in a control method of the portable medical device 1 below.

<Remote Controller 100>

The remote controller 100 operates the device body 10. In a case in which this portable medical device 1 is an insulin pump, the remote controller 100 is used for performing basal dosing of insulin by the device body 10, or optionally performing bolus dosing of insulin by the device body 10 in accordance with an operation by the user.

Such a remote controller 100 includes a power supply unit 101, an input unit 102a, a display unit 102b, a date and time management unit 103, a storage unit 104, a controller-side wireless communication unit 105, and a controller-side input/output control unit 106 that controls these. Details of each component are as follows.

[Power Supply Unit 101]

The power supply unit 101 is for supplying electric power to each component constituting the remote controller 100, and includes, for example, a battery and a battery box to accommodate the battery, as well as a switch that turns on and off the supply of electric power from the battery, and the like.

[Input Unit 102a]

The input unit 102a is a user interface part to which the user inputs an on/off operation for power supply, an administration condition of a drug solution by the device body 10, a stop setting of wireless output from the main-body-side wireless communication unit 15, and selection of display contents on the display unit 102b. Such an input unit 102a may be, for example, a keypad or a touch panel provided on a front surface of the display unit 102b. In a case in which the input unit 102a is a touch panel, the display unit 102b displays selection buttons, a ten key, and the like, for the touch panel.

The wireless output stop setting for the main-body-side wireless communication unit 15 inputted from the input unit 102a is a stop request of wireless output, and a stop duration (for example, three hours) of the output from stopping to restarting of the wireless output or date and time (time of day) for restarting the wireless output. In addition, the wireless output stop setting inputted from the input unit 102a may be a time for stopping the wireless output, and a time period (for example, three hours) from stopping to restarting the wireless output or a time for restarting the wireless output. In this case, the time for stopping the wireless output is to be the stop request of the wireless output.

[Display Unit 102b]

The display unit 102b displays various setting contents, input contents from the input unit 102a, a history of drug solution administration performed by the device body 10, and the like. Such a display unit 102b is configured with use of, for example, a liquid crystal display.

[Date and Time Management Unit 103 (Remote Controller 100)]

The date and time management unit 103 may be similar to the date and time management unit 13 of the device body 10, and is a program part to perform date and time management.

[Storage Unit 104]

The storage unit 104 is a part to store various data. Data stored in the storage unit 104 includes, for example, a history of drug solution administration transmitted from the device body 10 side, an administration condition of a drug solution inputted in the input unit 102a, a wireless output stop setting in the main-body-side wireless communication unit 15, and further, authentication data for communication connection with the device body 10, and the like. Note that the authentication data of communication connection may be identification information for recognizing the device body 10 in communication connection with this remote controller 100, and is also a link key created automatically by the communication connection.

[Controller-Side Wireless Communication Unit 105]

The controller-side wireless communication unit 105 is a part that performs wireless communication with the device body 10. This controller-side wireless communication unit 105 includes a transmission unit and a reception unit. This controller-side wireless communication unit 105 is applied with a standardized wireless communication technology similar to that of the main-body-side wireless communication unit 15.

Such a controller-side wireless communication unit 105 performs transmission and reception of signals with the main-body-side wireless communication unit 15 of the device body 10 in communication connection, in accordance with an instruction from the controller-side input/output control unit 106, and the controller-side wireless communication unit 105 establishes connection of wireless communication with the main-body-side wireless communication unit 15. Further, in accordance with an instruction from the controller-side input/output control unit 106, the controller-side wireless communication unit 105 transmits various settings inputted in the input unit 102a, to the main-body-side wireless communication unit 15 of the device body 10 in communication connection. Such a controller-side wireless communication unit 105 transmits a wireless output stop setting for the main-body-side wireless communication unit 15 inputted from the input unit 102a.

[Controller-Side Input/Output Control Unit 106]

The controller-side input/output control unit 106 is connected to the power supply unit 101, the input unit 102a, the display unit 102b, the date and time management unit 103, the storage unit 104, and the controller-side wireless communication unit 105. By controlling input and output of signals between these units, the controller-side input/output control unit 106 controls display on the display unit 102b and wireless communication by the controller-side wireless communication unit 105.

The controller-side input/output control unit 106 as described above includes, for example, a central processing unit (CPU), a random access memory (RAM), and a read only memory (ROM), which are not shown here. The read only memory (ROM) may be the storage unit 104. An algorithm for the control of wireless communication by the controller-side wireless communication unit 105 executed by such a controller-side input/output control unit 106 will be described in detail in the control method of the portable medical device 1 next.

<Control Method of Portable Medical Device 1>

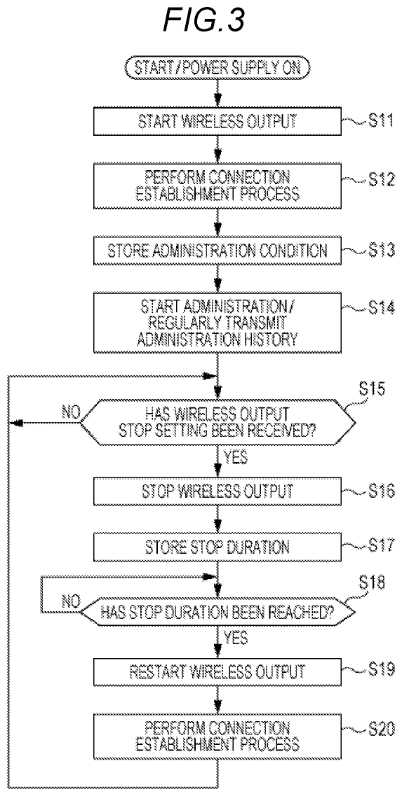

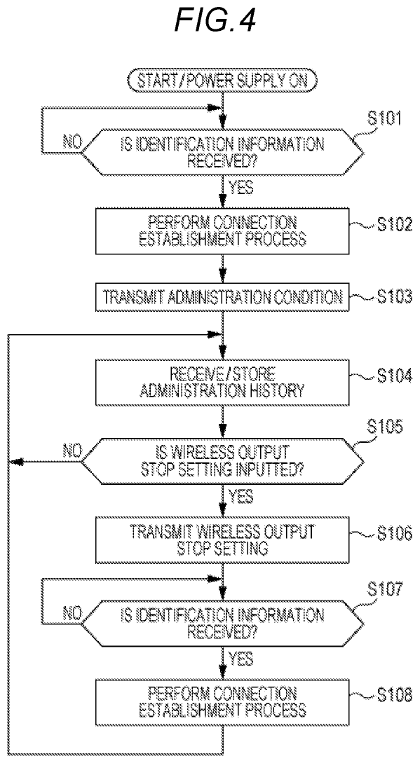

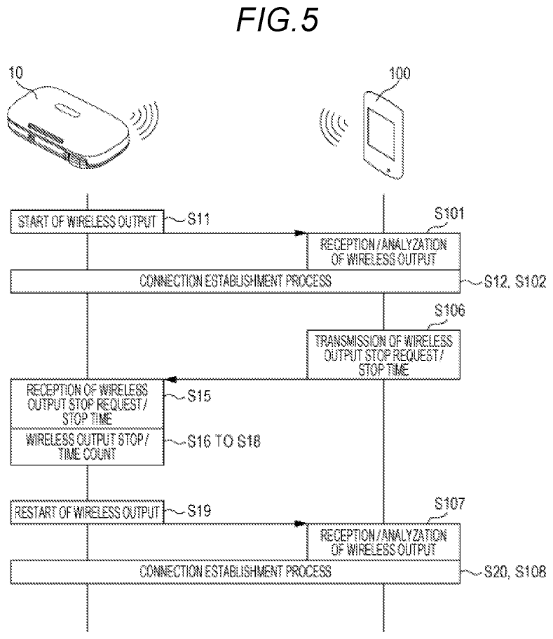

FIG. 3 is a flowchart (part 1) for explaining the control method of the portable medical device 1 of the first embodiment, and shows a procedure (steps S11 to S20) performed by the main-body-side input/output control unit 16 of the device body 10. Further, FIG. 4 is a flowchart (part 2) for explaining the control method of the portable medical device 1 of the first embodiment, and shows a procedure (steps S101 to S108) performed by the controller-side input/output control unit 106 of the remote controller 100. Furthermore, FIG. 5 is a time chart for explaining a state of wireless communication of the portable medical device 1 of the first embodiment.

The control method of the portable medical device 1 of the first embodiment described using these figures is a series of procedures in a case in which it is assumed that wireless communication between the device body 10 and the remote controller 100 is temporarily stopped during drug solution administration by the device body 10. The procedure described below is realized by a CPU constituting the main-body-side input/output control unit 16 of the device body 10 and a CPU constituting the controller-side input/output control unit 106 of the remote controller 100 executing a program recorded in each ROM and RAM.

Hereinafter, the control method of the portable medical device 1 will be described in the order shown in the flowcharts of FIGS. 3 and 4, with reference to FIGS. 2 and 5.

[Step S11 (FIG. 3)]

First, in step S11, when the power supply unit 11 is turned on by setting the battery in the battery box of the power supply unit 11, with this as a trigger, the main-body-side input/output control unit 16 starts wireless output from the main-body-side wireless communication unit 15. The main-body-side wireless communication unit 15 continuously transmits, as wireless output, identification information for identifying the device body 10 at a regular interval of, for example, 0.5 seconds.

[Step S101 (FIG. 4)]

In step S101, when the power supply unit 101 is turned on, for example, by an operation on the input unit 102a, with this as a trigger, the controller-side input/output control unit 106 starts signal reception by the controller-side wireless communication unit 105, and determines whether or not identification information from the main-body-side wireless communication unit 15 has been received. The controller-side input/output control unit 106 analyzes the wireless output received by the controller-side wireless communication unit 105. Then, it is determined whether or not the received wireless output is identification information of the device body 10 in communication connection with this remote controller 100. Then, the determination is repeated until it is determined that this identification information has been received (YES), and the process proceeds to the next step S102 when it is determined that the identification information has been received (YES).

[Step S102 (FIG. 4)]

Next, in step S102, the controller-side input/output control unit 106 performs a connection establishment process for establishing connection of wireless communication between the controller-side wireless communication unit 105 and the main-body-side wireless communication unit 15.

[Step S12 (FIG. 3)]

Similarly, in step S12, the main-body-side input/output control unit 16 performs a connection establishment process for establishing connection of wireless communication between the controller-side wireless communication unit 105 and the main-body-side wireless communication unit 15.

[Step S103 (FIG. 4)]

Thereafter, in step S103, the controller-side input/output control unit 106 causes the controller-side wireless communication unit 105 to transmit an administration condition of a drug solution by the driving unit 12 of the device body 10. This administration condition is information inputted by the user from the input unit 102a or information inputted by the user from the input unit 102a and stored in the storage unit 104.

[Step S13 (FIG. 3)]

In step S13, the main-body-side input/output control unit 16 stores the administration condition of the drug solution transmitted from the controller-side wireless communication unit 105 and received by the main-body-side wireless communication unit 15, in the storage unit 14 as a set value.

[Step S14 (FIG. 3)]

Thereafter, in step S14, the main-body-side input/output control unit 16 controls the driving unit 12 in accordance with the administration condition stored in the storage unit 14, to start drug solution administration. Further, when performing drug solution administration, the main-body-side input/output control unit 16 stores an administration history of the drug solution actually administered by the control of the driving unit 12, in the storage unit 14. Furthermore, the main-body-side input/output control unit 16 causes the main-body-side wireless communication unit 15 to regularly transmit the administration history of the drug solution stored in the storage unit 14, to the controller-side wireless communication unit 105.

[Step S104 (FIG. 4)]

In step S104, when the administration history transmitted from the main-body-side wireless communication unit 15 has been received by the controller-side wireless communication unit 105, the controller-side input/output control unit 106 stores the received administration history in the storage unit 104, and causes the display unit 102b to display as necessary.

The above procedure is a procedure of a normal drug solution administration by the portable medical device 1. Among these, step S14 performed by the device body 10 and step S104 performed by the remote controller 100 are continuously performed in accordance with the setting of the administration condition of the drug solution, stored in the storage unit 14 of the device body 10.

Then, a procedure described below is to be a procedure unique to the first embodiment. The procedure described below is a procedure in a case in which it becomes necessary to stop wireless communication between the device body 10 and the remote controller 100 during execution of the normal drug solution administration, for example, such as a case in which the user of the portable medical device 1 boards an aircraft in which in-flight wireless communication is prohibited, and the procedure is performed as follows.

[Step S105 (FIG. 4)]

First, in step S105, the controller-side input/output control unit 106 determines whether or not a wireless output stop setting has been inputted from the input unit 102a. Then, when it is determined that there is an input (YES), the process proceeds to the next step S106, whereas, when it is determined that there is no input (NO), the process returns to step S104.

Note that the determination as to whether or not a wireless output stop setting by the controller-side input/output control unit 106 has been inputted may be determination based on whether or not it is time for stopping the wireless output on the basis of setting information for stopping wireless output stored in advance in the storage unit 104.

[Step S106 (FIG. 4)]

Next, in step S106, the controller-side input/output control unit 106 causes the controller-side wireless communication unit 105 to transmit a wireless output stop setting. The wireless output stop setting to be transmitted here is a stop request of wireless output and a length of a time period (for example, three hours) from stopping to restarting of the wireless output, or a time for starting the wireless output.

[Step S15 (FIG. 3)]

In step S15, the main-body-side input/output control unit 16 determines whether or not a wireless output stop setting has been received in the main-body-side wireless communication unit 15 and repeats the determination until it is determined as being received (YES), and the process proceeds to the next step S16 when it is determined as being received (YES).

[Step S16 (FIG. 3)]

Next, in step S16, the main-body-side input/output control unit 16 stops the wireless output of the main-body-side wireless communication unit 15. This interrupts the wireless connection between the main-body-side wireless communication unit 15 and the controller-side wireless communication unit 105 established in step S12 and step S102. In this state, in order to restart the wireless connection between the main-body-side wireless communication unit 15 and the controller-side wireless communication unit 105, it is necessary to perform the connection establishment process again.

[Step S17 (FIG. 3)]

Further, in step S17, the main-body-side input/output control unit 16 stores, among the wireless output stop setting received in step S15, the length of the time period (for example, three hours) from stopping to restarting of the wireless output or the time for restarting the wireless output, in the storage unit 14.

[Step S18 (FIG. 3)]

Further, in step S18, the main-body-side input/output control unit 16 determines whether or not a stop duration or a restart time after stopping of the wireless output of the main-body-side wireless communication unit 15 has reached a set time period (or time of day) stored in the storage unit 14. It is determined whether or not the time managed by the date and time management unit 13 corresponds to the length of the time period for stopping the wireless output stored in the storage unit 14 after stopping the wireless communication, or whether or not the time for restarting the wireless output has been reached.

Then, the determination is repeated until it is determined as being reached (YES), and the process proceeds to the next step S19 when it is determined as being reached (YES).

[Step S19 (FIG. 3)]

Next, in step S19, the main-body-side input/output control unit 16 restarts the wireless output from the main-body-side wireless communication unit 15. The main-body-side wireless communication unit 15 continuously transmits, as wireless output, identification information for identifying the device body 10 at a regular interval of, for example, 0.5 seconds.

[Step S107 (FIG. 4)]

In step S107, the controller-side input/output control unit 106 determines whether or not the controller-side wireless communication unit 105 has received identification information. When the controller-side wireless communication unit 105 has received wireless output, the controller-side input/output control unit 106 analyzes the wireless output. Then, it is determined whether or not the received wireless output is identification information of the device body 10 in communication connection with this remote controller 100. Then, the determination is repeated until it is determined that this identification information has been received (YES), and the process proceeds to the next step S108 when it is determined that the identification information has been received (YES).

[Step S108 (FIG. 4)]

Next, in step S108, the controller-side input/output control unit 106 performs a connection establishment process for establishing connection of wireless communication between the controller-side wireless communication unit 105 and the main-body-side wireless communication unit 15, and thereafter, the process returns to step S104.

[Step S20 (FIG. 3)]

Similarly, in step S20, the main-body-side input/output control unit 16 performs a connection establishment process for establishing connection of wireless communication between the controller-side wireless communication unit 105 and the main-body-side wireless communication unit 15, and thereafter, the process returns to step S15.

### Effect of First Embodiment

According to the first embodiment described above, for example, when the user boards an aircraft in which in-flight wireless communication is prohibited, wireless output can be automatically restarted after the lapse of a wireless stop duration or a restart time even when the wireless output of the device body 10 having no user interface is stopped by an operation of the remote controller 100. Therefore, it becomes possible again to change the setting of drug solution administration, and to perform additional drug solution administration, that is, bolus administration of insulin from the device body 10.

### Second Embodiment

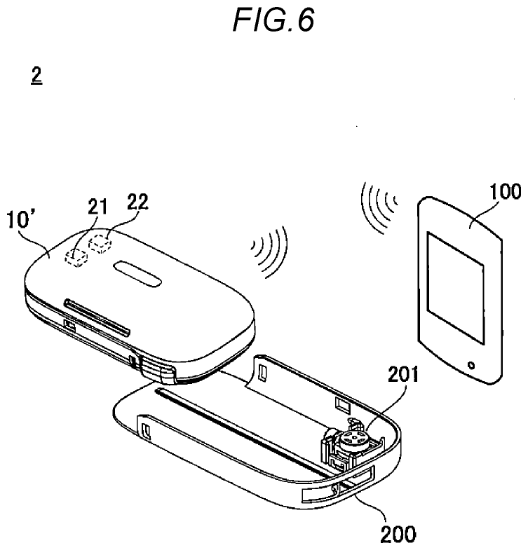

FIG. 6 is a schematic configuration view of a portable medical device 2 of a second embodiment. The portable medical device 2 of the second embodiment shown in FIG. 6 is different from the portable medical device 1 of the first embodiment described above in that a device body 10′ includes an attachment detection unit 21 and a blockage detection unit 22 as a drive state detection unit. Further, a procedure of control performed by a main-body-side input/output control unit 16′ described later is different. Other configurations are similar to those in the first embodiment. Therefore, in the following, components similar to those in the first embodiment are denoted by the same reference numerals, and only configurations different from the first embodiment will be described while redundant description will be omitted.

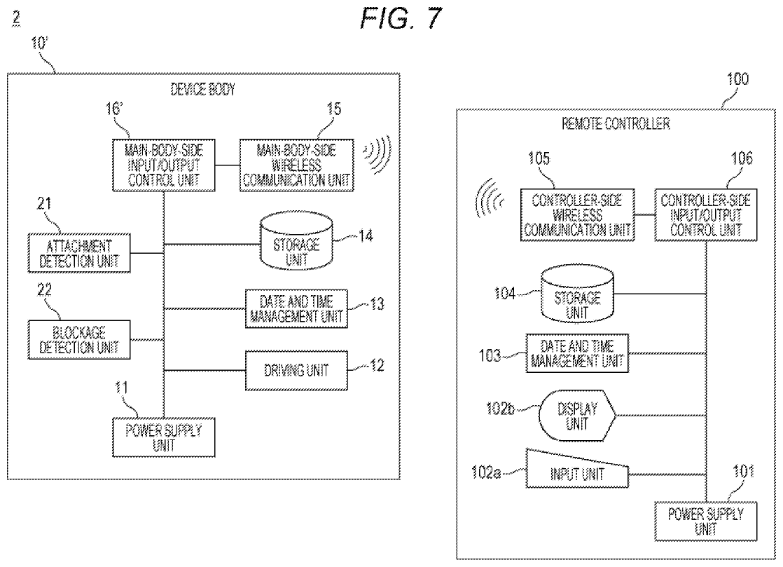

FIG. 7 is a block diagram for explaining the portable medical device of the second embodiment. Hereinafter, with reference to FIG. 7, features of a configuration of the device body 10′ of the portable medical device 2 will be described.

<Device Body 10′>

In the device body 10′, functions for performing administration of a drug solution (insulin as an example) are intensively arranged. Such a device body 10′ includes a power supply unit 11, a driving unit 12, a date and time management unit 13, a storage unit 14, and a main-body-side wireless communication unit 15 similar to those of the first embodiment, and further includes the attachment detection unit 21 and the blockage detection unit 22. Further, the device body 10′ includes the main-body-side input/output control unit 16′ that controls these. Among them, the attachment detection unit 21, the blockage detection unit 22, and the main-body-side input/output control unit 16′ having a configuration different from that of the first embodiment are as follows.

[Attachment detection unit 21 (Device body 10′)] The attachment detection unit 21 is for detecting whether or not the device body 10′ is attached to a cradle 200. As an example of such an attachment detection unit 21, for example, one is exemplified that includes a light emitting element and a light receiving element that detects reflected light of light emitted from the light emitting element. Such an attachment detection unit 21 has a configuration in which, in a state in which the device body 10′ is attached to the cradle 200, light emitted from the light emitting element is reflected by the cradle 200 and detected by the light receiving element, and whereby it is detected that the device body 10′ is attached to the cradle 200.

Further, in particular, the attachment detection unit 21 also serves as input means in the device body 10′. That is, the attachment detection unit 21 switches on and off of a detection state of reflected light in the light receiving element by switching of attachment/non-attachment of the device body 10′ to the cradle 200, that is, by attachment and removal of the device body 10′ to and from the cradle 200. Therefore, for example, with a speed of this on/off switching, predetermined processing related to control of the main-body-side wireless communication unit 15 is associated. This association is stored in the storage unit 14.

For example, one switching operation of removing the device body 10′ in a state of being attached to the cradle 200 to set in an non-attached state and attaching again within a predetermined time period (10 seconds) is associated with processing of extending a stop duration or a restart time of the wireless output of the main-body-side wireless communication unit 15, by a predetermined extension time (for example, 10 minutes).

Further, two switching operations within a predetermined time period (10 seconds) are associated with a trigger for forcibly restarting the wireless output of the main-body-side wireless communication unit 15.

Note that the attachment detection unit 21 is not limited to one that includes the light emitting element and the light receiving element as described above, and may be, for example, one that detects an attachment state of the device body 10′ to the cradle 200 by mechanical switching, a reed switch, or the like.

[Blockage Detection Unit 22 (Device Body 10′)]

The blockage detection unit 22 is a drive state detection unit for detection of a drive failure of the driving unit 12. For example, in a case in which the driving unit 12 includes a syringe to contain a drug solution to be administered, a pusher to be slid in the syringe, a motor and gear mechanism for movement of the pusher, and the like, this blockage detection unit 22 may be an encoder provided in the gear mechanism, as an example. In addition, the blockage detection unit 22 may detect a drive state by a pressure sensor or a flow rate sensor.

[Main-Body-Side Input/Output Control Unit 16′ (Device Body 10′)]

The main-body-side input/output control unit 16′ is connected to the power supply unit 11, the driving unit 12, the date and time management unit 13, the storage unit 14, the main-body-side wireless communication unit 15, the attachment detection unit 21, and the blockage detection unit 22. This main-body-side input/output control unit 16′ controls input and output of signals between these units, and controls drug solution administration by driving of the driving unit 12 and wireless communication by the main-body-side wireless communication unit 15, in accordance with information received by the main-body-side wireless communication unit 15 and signals from the attachment detection unit 21 and the blockage detection unit 22.

The fact that such a main-body-side input/output control unit 16′ includes, for example, a central processing unit (CPU), a random access memory (RAM), and a read only memory (ROM), which are not shown here, is similar to the first embodiment.

Next, an algorithm executed by such a main-body-side input/output control unit 16′ for the control of drug solution administration from the driving unit 12 and wireless communication by the main-body-side wireless communication unit 15 will be described as a control method of the portable medical device 2.

<Control Method of Portable Medical Device 2>

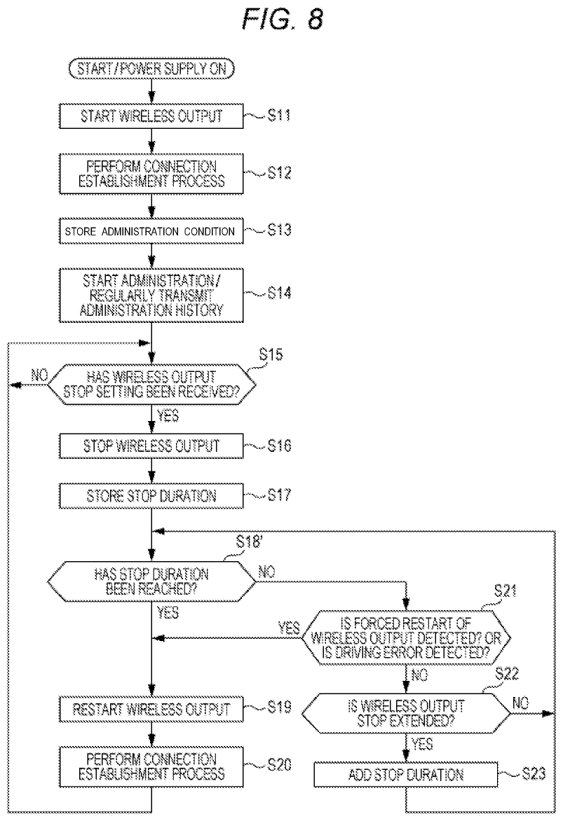

FIG. 8 is a flowchart for explaining the control method of the portable medical device 2 of the second embodiment, and shows a procedure to be performed by the main-body-side input/output control unit 16′ of the device body 10′. A difference between the procedure shown in FIG. 8 and the procedure to be performed by the main-body-side input/output control unit 16 of the device body 10 of the first embodiment described using FIG. 3 is that steps S21 to S23 are added after the determination in step S18′. Other steps S11 to S20 are similar to the procedure described in the first embodiment. Further, a procedure to be performed by the controller-side input/output control unit 106 of the remote controller 100 is similar to steps S101 to S108 described using FIG. 4. Hereinafter, with reference to FIG. 7, a procedure different from that of the first embodiment will be described with reference to FIG. 8.

[Step S18′ (FIG. 8)]

In step S18′, the main-body-side input/output control unit 16′ determines whether or not a stop duration or a restart time after stopping of the wireless output of the main-body-side wireless communication unit 15 has reached a stop duration or a restart time stored in the storage unit 14. This determination is performed similarly to step S18 in the first embodiment, and the process proceeds to the next step S19 similarly to the first embodiment when it is determined as having reached (YES).

When the main-body-side input/output control unit 16′ determines that the stop duration or the restart time after stopping of the wireless output of the main-body-side wireless communication unit 15 has not reached the stop duration or the restart time stored in the storage unit 14 (NO), the process proceeds to step S21.

[Step S21 (FIG. 8)]

In step S21, the main-body-side input/output control unit 16′ makes two determinations as to whether or not forced restart of wireless output has been detected, and whether or not an error contrary to a setting condition has occurred in drug solution administration from the driving unit 12. The error includes blockage of a drug solution administration route, lack of a remaining drug solution, and the like.

That is, the main-body-side input/output control unit 16′ determines whether or not there is detection associated with forced restart of the wireless output, in the attachment detection unit 21. For example, it is determined whether or not an operation for removing the device body 10′ in a state of being attached to the cradle 200 from the cradle 200 and attaching again has been detected twice within a predetermined time period (10 seconds) in the attachment detection unit 21.

Further, the main-body-side input/output control unit 16′ determines whether or not an occurrence of blockage in an administration route of the drug solution from the driving unit 12 has been detected in the blockage detection unit 22.

When it is determined that either one is detected (YES), the process proceeds to step S19, the wireless output from the main-body-side wireless communication unit 15 is restarted, and identification information for identifying the device body 10′ is continuously transmitted at a regular interval of, for example, 0.5 seconds. Further, in the next step S20, a connection establishment process of wireless communication between the main-body-side wireless communication unit 15 and the controller-side wireless communication unit 105 is performed.

Then, although illustration is omitted here, when the blockage detection unit 22 has detected that blockage has occurred in an administration route of the drug solution from the driving unit 12, blockage occurrence information is transmitted from the main-body-side wireless communication unit 15 to the remote controller 100 in accordance with a normal procedure. With regard to notification of the blockage, a notification unit may be provided in the device body 10′, and notification may be made by an alarm sound or vibration. This has the effect that the user can quickly recognize the abnormality. Also in this case, even without using the remote controller 100, it becomes possible to input a postponement or stop of communication restart of the device body 10′ by an operation of the attachment detection unit 21.

When the main-body-side input/output control unit 16′ determines that none is detected in step S21 (NO), the process proceeds to step S22.

[Step S22 (FIG. 8)]

In step S22, the main-body-side input/output control unit 16′ determines whether or not extension of wireless output has been detected. The main-body-side input/output control unit 16′ determines whether or not there is detection associated with extension of wireless output, in the attachment detection unit 21. For example, it is determined whether or not an operation for removing the device body 10′ in a state of being attached to the cradle 200 from the cradle 200 and attaching again has been detected once within a predetermined time period (10 seconds) in the attachment detection unit 21.

When it is determined as being detected (YES), the process proceeds to step S23, whereas, when it is determined as not being detected (NO), the process returns to step S18′.

[Step S23 (FIG. 8)]

In step S23, the main-body-side input/output control unit 16′ performs a process of adding a stop duration of the wireless output (or a time for restarting the wireless output) stored in the storage unit 14. The stop duration of the wireless output (or the time for restarting the wireless output) stored in the storage unit 14 is rewritten to a time extended by a predetermined extension time (for example, 10 minutes). Thereafter, the process returns to step S18′ to continue processing.

### Effect of Second Embodiment

According to the second embodiment described above, in addition to the effects of the first embodiment, in a state in which the wireless output of the device body 10 having no user interface is stopped, it is possible forcibly restart the wireless output of the device body 10′ and extend the stop of the wireless output, even without operation of the remote controller. Therefore, for example, when the user of the portable medical device 2 is on board an aircraft in which in-flight wireless communication is prohibited, the stop of the wireless output can be extended if arrival of the aircraft is delayed. In addition, even when an unexpected situation related to drug solution administration occurs while the wireless output is stopped, the wireless output of the device body 10 can be restarted urgently to enable the remote controller 100 to operate the device body 10.

It should be noted that the present invention is not limited to the embodiments described above and shown in the drawings, and various modifications can be made without departing from the subject matter of the invention described in the claims.

### REFERENCE NUMERAL LIST

1, 2 portable medical device
10, 10′ device body
11 power supply unit
12 driving unit
15 main-body-side wireless communication unit
16 main-body-side input/output control unit
21 attachment detection unit
22 blockage detection unit (drive state detection unit)
100 remote controller
102a input unit
105 controller-side wireless communication unit

1. A portable medical device comprising:
a device body comprising:
a power supply unit,
a driving unit configured to be driven by electric power from the power supply unit,
a main-body-side wireless communication unit configured to output identification information at a regular interval when the power supply unit is turned on, and
a main-body-side input/output control unit configured to control driving of the driving unit and the output of the main-body-side wireless communication unit, in accordance with information received by the main-body-side wireless communication unit; and
a remote controller configured to operate driving of the device body, the remote controller comprising:
an input unit configured to receive a stop request for a stop of the output from the main-body-side wireless communication unit, and at least one of a stop duration of the stop and/or a restart time at which the output is to be restarted, and
a controller-side wireless communication unit configured to:
establish a wireless connection with the main-body-side wireless communication unit that has outputted the identification information by receiving the identification information from the main-body-side wireless communication unit, and
output, to the main-body-side wireless communication unit, the stop request and said at least one of the stop duration and/or the restart time.

2. The portable medical device according to claim 1, wherein:
the main-body-side input/output control unit is configured to:
stop the output of the identification information from the main-body-side wireless communication unit in accordance with the stop request received by the main-body-side wireless communication unit from the controller-side wireless communication unit, and
restart the output of the identification information from the main-body-side wireless communication unit when the stop duration of the stop has passed and/or when the restart time of the output has been reached.

3. The portable medical device according to claim 1, wherein:
the device body is configured to be used in a predetermined attachment state on a user, and comprises an attachment detection unit that detects an attachment/non-attachment state of the device body on the user; and
the main-body-side input/output control unit is configured such that, upon switching of the attachment/non-attachment state of the device body, as detected by the attachment detection unit, the main-body-side input/output control unit causes the main-body-side wireless communication unit that is in a state in which the output of the identification information is stopped to switch to a state in which the output of the identification information is restarted.

4. The portable medical device according to claim 1, wherein:
the device body is configured to be used in a predetermined attachment state on a user, and comprises an attachment detection unit that detects an attachment/non-attachment state of the device body on the user; and
the main-body-side input/output control unit is configured such that, upon switching of the attachment/non-attachment state of the device body, as detected by the attachment detection unit, the main-body-side input/output control unit extends the stop duration or delays the restart time.

5. The portable medical device according to claim 1, wherein:
the device body is configured to be used in a predetermined attachment state on a user, and comprises an attachment detection unit that detects an attachment/non-attachment state of the device body on the user; and
the main-body-side input/output control unit is configured to control the output of the main-body-side wireless communication unit by the main-body-side input/output control unit in different ways depending on a speed at which switching of the attachment/non-attachment state of the device body occurs, as detected by the attachment detection unit.

6. The portable medical device according to claim 1, wherein:
the device body comprises a drive state detection unit configured to detect a drive failure of the driving unit; and
the main-body-side input/output control unit is configured such that, when the drive state detection unit detects a drive failure of the driving unit, the main-body-side input/output control unit causes the main-body-side wireless communication unit that is in a state in which the output of the identification information is stopped to switch to a state in which the output of the identification information is restarted.

7. A portable medical device comprising:
a device body comprising:
a power supply unit comprising a battery,
a driving unit configured to be driven by electric power from the power supply unit, the driving unit comprising a syringe configured to contain a drug solution to be administered, a pusher to be slid in the syringe, and a motor and gear mechanism configured to move the pusher;
a main-body-side wireless communication unit configured to output identification information at a regular interval when the power supply unit is turned on, the main-body-side wireless communication unit comprising a transmitter and a receiver, and
a main-body-side input/output control unit configured to control driving of the driving unit and the output of the main-body-side wireless communication unit, in accordance with information received by the main-body-side wireless communication unit, the main-body-side input/output control unit comprising a central processing unit, a random access memory, and a read only memory; and
a remote controller configured to operate driving of the device body, the remote controller comprising:
an input unit configured to receive a stop request for a stop of the output from the main-body-side wireless communication unit, and at least one of a stop duration of the stop and/or a restart time at which the output is to be restarted, the input unit comprising a keypad or a touch panel, and
a controller-side wireless communication unit comprising a transmitter and a receiver and configured to:
establish a wireless connection with the main-body-side wireless communication unit that has outputted the identification information by receiving the identification information from the main-body-side wireless communication unit, and
output, to the main-body-side wireless communication unit, the stop request and said at least one of the stop duration and/or the restart time.

8. A method for controlling a portable medical device, the method comprising:
providing a portable medical device comprising:
a device body comprising:
a power supply unit,
a driving unit driven by electric power from the power supply unit,
a main-body-side wireless communication unit, and
a main-body-side input/output control unit; and
a remote controller that operates driving of the device body, the remote controller comprising:
an input unit, and
a controller-side wireless communication unit;
outputting, from the main-body-side wireless communication unit, identification information at a regular interval when the power supply unit is turned on;
receiving, at the input unit, a stop request for a stop of the output from the main-body-side wireless communication unit, and at least one of a stop duration of the stop and/or a restart time at which the output is to be restarted;
establishing, with the controller-side wireless communication unit, a wireless connection with the main-body-side wireless communication unit that has outputted the identification information by receiving the identification information from the main-body-side wireless communication unit;
outputting, from the controller-side wireless communication unit to the main-body-side wireless communication unit, the stop request and said at least one of the stop duration and/or the restart time;
stopping, using the main-body-side input/output control unit, the output of the identification information from the main-body-side wireless communication unit in accordance with the stop request received by the main-body-side wireless communication unit from the controller-side wireless communication unit; and
restarting, using the main-body-side input/output control unit, the output of the identification information from the main-body-side wireless communication unit when the stop duration of the stop has passed and/or when the restart time of the output has been reached.
